(19) United States
(12) Patent Application Publication
(10) Pub. No.: US 2022/0280166 A1
(43) Pub. Date: Sep. 8, 2022

(54) OCCLUDER STABILIZING MEMBERS

(71) Applicant: St. Jude Medical, Cardiology Division, Inc., St. Paul, MN (US)

(72) Inventors: Kristen Morin, Saint Paul, MN (US); Trevor Springer, Stillwater, MN (US); Andrey Samaray, Champlin, MN (US); Linda Cornelius, Wayzata, MN (US); Tracee Eidenschink, Wayzata, MN (US); Yiru Wang, Minneapolis, MN (US); Christian Elmquist, Minneapolis, MN (US); Sam S Leutbounshu, Lakeville, MN (US)

(21) Appl. No.: 17/684,183

(22) Filed: Mar. 1, 2022

Related U.S. Application Data
(60) Provisional application No. 63/156,093, filed on Mar. 3, 2021.

(51) Int. Cl. A61B 17/12; A61B 90/00

(52) U.S. Cl. CPC A61B 17/12177; A61B 90/03; A61B 2017/00867

(57) ABSTRACT

A medical device including stabilizing members and a delivery system including the same are described herein. The medical device includes a device body and the stabilizing members coupled thereto. Each stabilizing member has a backing portion coupled to the device body and an engagement portion extending from an outward face of the backing portion. The engagement portion is configured to extend radially outward from the device body. The medical device also includes one or more features configured to control at least one of tissue penetration depth of the at least one engagement portion, extension of the stabilizing member from the device body, and tissue engagement of the at least one engagement portion. The delivery system includes the medical device and a delivery sheath configured to retain and recapture the medical device during deployment to a target site.

### CROSS-REFERENCE TO RELATED APPLICATIONS

This application claims the benefit of priority to U.S. Prov. Pat. App. No. 63/156,093 filed Mar. 3, 2021, which is incorporated by reference herein in its entirety.

### BACKGROUND OF THE DISCLOSURE

### A. Field of Disclosure

The present disclosure relates generally to medical devices that are used in the human body. In particular, the present disclosure is directed to stabilizing members incorporated into medical devices that are delivered to a target site within the human body. More specifically, the present disclosure is directed to stabilizing members that may reduce damage to cardiac tissue and for which tissue penetration depth can be controlled.

### B. Background

A wide variety of medical devices are used to treat any target site, such as an abnormality, a vessel, an organ, an opening, a chamber, a channel, a hole, a cavity, or the like, located anywhere in the body. Some conventional medical devices include conventional wires 12 (FIG. 1), which extend outward from a body of the medical device. The presence of stabilizing members (formed from wire or other materials described herein) as an alternative to conventional wires 12 may decrease the risk of the medical device migrating from its deployed location over time.

Once medical devices with conventional wires 12 are deployed, the length of the conventional wires 12 may provide adequate engagement of surrounding tissue and prevent the device from becoming dislodged. In some instances however, the curve of the wire (absent any additional depth control feature or component) may provide less than desired control over penetration depth into the tissue. Specifically, the conventional wires 12 may penetrate the tissue too deeply in some cases and cause problematic issues such as cardiac tissue damage and pericardial effusion.

For example, left atrial appendage (LAA) closure devices have gained traction in the treatment of patients with atrial fibrillation. Some conventional wire designs include two wire legs connected in a U shape, each with a hook at the distal aspect (FIG. 2 and FIG. 3). The conventional wire is attached to the braid with a suture stitch on each leg. In some conventional designs, conventional wires 12 may be relatively long and if their penetration depth into the tissue is not adequately controlled, the wire legs may be able to slide through the stitch and over-extend outward from a lobe of the device body, such as if the lobe of the device body is compressed axially (FIG. 2). Inadequate control over tissue penetration depth can potentially cause perforation of the LAA, which may cause pericardial effusion or tamponade.

### SUMMARY OF THE DISCLOSURE

In one embodiment, the present disclosure is directed to a medical device for treating a target site. The medical device includes a device body including at least one disc formed from a shape memory material, and a plurality of stabilizing members coupled to the device body. Each stabilizing member includes a backing portion coupled to the device body, an engagement portion extending from an outward face of the backing portion, and one or more features configured to control at least one of tissue penetration depth of the at least one engagement portion, extension of the stabilizing member from the device body, and tissue engagement of the at least one engagement portion. The engagement portion extends radially outward from the device body.

In another embodiment, the present disclosure is directed to a delivery system including a medical device and a delivery sheath. The medical device includes a device body including at least one disc formed from a shape memory material, and a plurality of stabilizing members coupled to the device body. Each stabilizing member includes a backing portion coupled to the device body, and an engagement portion extending from an outward face of the backing portion. The engagement portion extends radially outward from the device body. The medical device also includes one or more features configured to control at least one of tissue penetration depth of the at least one engagement portion, extension of the stabilizing member from the device body, and tissue engagement of the at least one engagement portion. The delivery sheath is configured to retain and recapture the medical device during deployment of the medical device to a target site.

In yet another embodiment, the present disclosure is directed to a method of attaching a stabilizing member to a medical device. The method comprises injection molding the stabilizing member directly to a device body of the medical device such that an engagement portion of the stabilizing member extends radially outward from the device body.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is an embodiment of a conventional medical device including conventional wires.

FIG. 2 is an embodiment of conventional wire extension from the lobe of a conventional occlusion device under partial axial compression.

FIG. 3 is an embodiment of conventional wire design.

FIG. 4A depicts a conventional wire shown exiting the braid for a conventional device. FIG. 4B depicts an optimized stabilizing member shown exiting the braid for an optimized device in accordance with the present disclosure.

FIG. 5A depicts a hooked engagement portion of a stabilizing member in accordance with the present disclosure. FIG. 5B depicts a comparison between a conventional wire and an exemplary embodiment of a stabilizing member with a shallower hook angle in accordance with the present disclosure.

FIG. 6A is comparison between a conventional wire design (right) compared to an exemplary embodiment of a stabilizing member having a small angle design (left) in accordance with the present disclosure. FIG. 6B is another comparison between a conventional wire design compared to an exemplary embodiment of a stabilizing member having small angle design and smaller hook radius in accordance with the present disclosure.

FIG. 7 depicts a comparison between a conventional wire and an exemplary embodiment of a stabilizing member with a smaller hook radius in accordance with the present disclosure.

FIG. 8 depicts a comparison between a conventional design (left) and an exemplary embodiment of a small hook radius (right) in accordance with the present disclosure.

FIG. 9 depicts exemplary embodiments of engagement portions of a stabilizing member having one or more hooks in accordance with the present disclosure.

FIG. 10A is an exemplary embodiment of tissue penetration depth limit of a stabilizing member in accordance with the present disclosure. FIG. 10B depicts a conventional wire having no limit to tissue penetration depth.

FIG. 11 is an exemplary embodiment of a complex geometry hooked engagement portion of a stabilizing member in accordance with the present disclosure.

FIG. 12 is an exemplary embodiment of a stabilizing member that is laser cut from a tubular material in accordance with the present disclosure.

FIG. 13A is an exemplary embodiment of a stabilizing member with a tail rotation restraint to prevent engagement portion rotation in accordance with the present disclosure. FIG. 13B is another exemplary embodiment of a stabilizing member with a tail rotation restraint to prevent engagement portion rotation in accordance with the present disclosure.

FIG. 14A is an exemplary embodiment of laser-cut paired stabilizing members to prevent engagement portion rotation in accordance with the present disclosure. FIG. 14B is an exemplary embodiment of the paired stabilizing members shown in FIG. 14A in a “U” shape in accordance with the present disclosure.

FIG. 15 is an exemplary embodiment of a wide split hook design cut from flat sheet nitinol in accordance with the present disclosure.

FIG. 16 is an exemplary embodiment of a narrow split hook design cut from flat sheet nitinol in accordance with the present disclosure.

FIG. 17 is an exemplary embodiment of a depth guard split hook design cut from flat sheet nitinol in accordance with the present disclosure.

FIG. 18 depicts a maximum extension length of a conventional wire design.

FIG. 19A is an exemplary embodiment of a stabilizing member with an eyelet in accordance with the present disclosure. FIG. 19B is an exemplary embodiment of the stabilizing member shown in FIG. 19A when deployed, having a maximum extension from the braid in accordance with the present disclosure. FIG. 19C is an exemplary embodiment of a stabilizing member with at least one eyelet formed by wire in accordance with the present disclosure. FIG. 19D is another exemplary embodiment of a stabilizing member with at least one eyelet formed by wire in accordance with the present disclosure. FIG. 19E is an exemplary embodiment of a stabilizing member with at least one eyelet formed by wire or by laser cut design in accordance with the present disclosure. FIG. 19F is another exemplary embodiment of a stabilizing member with at least one eyelet formed by wire or by laser cut design in accordance with the present disclosure. FIG. 19G is an exemplary embodiment of a stabilizing member with a base loop rotation restraint to prevent engagement portion rotation in accordance with the present disclosure. FIG. 19H is another exemplary embodiment of a stabilizing member with a base loop rotation restraint to prevent engagement portion rotation in accordance with the present disclosure. FIG. 19I is an exemplary embodiment of a stabilizing member with a T-bar rotation restraint to prevent engagement portion rotation in accordance with the present disclosure.

FIG. 20A is an exemplary embodiment of a stabilizing member with eyelet alternative in accordance with the present disclosure. FIG. 20B is another exemplary embodiment of a stabilizing member with eyelet alternative in accordance with the present disclosure. FIG. 20C is yet another exemplary embodiment of a stabilizing member with eyelet alternative in accordance with the present disclosure.

FIG. 21A is an exemplary embodiment of a stabilizing member with a feature to catch behind the braid in accordance with the present disclosure. FIG. 21B is an exemplary embodiment of the stabilizing member shown in FIG. 21A when deployed, having a maximum extension from the braid in accordance with the present disclosure.

FIG. 22A is an exemplary embodiment of a stabilizing member design with multiple hooks in accordance with the present disclosure. FIG. 22B is an exemplary embodiment of the stabilizing member design shown in FIG. 22A when deployed, having a maximum extension from the braid in accordance with the present disclosure. FIG. 22C is another exemplary embodiment of a stabilizing member design with multiple hooks in accordance with the present disclosure. FIG. 22D is an exemplary embodiment of the stabilizing member design shown in FIG. 22C when deployed, having a maximum extension from the braid in accordance with the present disclosure.

FIG. 23A is an exemplary embodiment of a stabilizing member having a crossed design in accordance with the present disclosure. FIG. 23B is an exemplary embodiment of the stabilizing member shown in FIG. 23A when deployed, enabling rotation upon axial compression in accordance with the present disclosure.

FIG. 24A is an exemplary embodiment of a stent-like stabilizing member in accordance with the present disclosure. FIG. 24B is an exemplary embodiment of the stent-like stabilizing member shown in FIG. 24A when deployed, having a maximum extension from the braid in accordance with the present disclosure.

FIG. 25A depicts a conventional wire leg length compared to an exemplary embodiments of a shortened leg stabilizing member in accordance with the present disclosure.

FIG. 25B is an exemplary embodiment of the shortened leg stabilizing member shown in FIG. 25A when deployed, having a shortened maximum extension from the braid in accordance with the present disclosure.

FIG. 26A is an exemplary embodiment of a stabilizing member with engagement portions at different axial positions in accordance with the present disclosure. FIG. 26B is an exemplary embodiment of applying multiple rows of hooks to the braid of an occlusion device in accordance with the present disclosure. FIG. 26C is another exemplary embodiment of applying multiple rows of hooks to the braid of an occlusion device in accordance with the present disclosure.

FIG. 27A is an exemplary embodiment of an injection-molded stabilizing member having a single engagement portion in accordance with the present disclosure. FIG. 27B is an exemplary embodiment of an injection-molded stabilizing member having multiple engagement portions in accordance with the present disclosure.

FIG. 28A is an exemplary embodiment of stabilizing member attachment to a device body in accordance with the present disclosure. FIG. 28B is another exemplary embodiment of stabilizing member attachment to a device body in accordance with the present disclosure.

FIG. 29 is an exemplary embodiment of a stabilizing member with a barbed engagement portion in accordance with the present disclosure.

FIG. 30 is an exemplary embodiment of a stabilizing member with a displaceable engagement portion in accordance with the present disclosure.

FIG. 31A is an exemplary embodiment of sewn-on placement of stabilizing members onto a device body in accordance with the present disclosure. FIG. 31B is another exemplary embodiment of sewn-on placement of stabilizing members onto a device body in accordance with the present disclosure.

FIG. 32A is an exemplary embodiment of injection-molded placement of stabilizing members onto a device body in accordance with the present disclosure. FIG. 32B is another exemplary embodiment of injection-molded placement of stabilizing members onto a device body in accordance with the present disclosure.

Corresponding reference characters indicate corresponding parts throughout the several views of the drawings. It is understood that that Figures are not necessarily to scale.

### DETAILED DESCRIPTION OF THE DISCLOSURE

The present disclosure generally relates to stabilizing members incorporated into medical devices for treating a target site. The present disclosure more specifically discloses medical devices having stabilizing members including an engagement portion, which extend radially outward from the body of the medical device to engage tissue at the target site at a controlled penetration depth.

It would be desirable to incorporate stabilizing members (e.g., formed from wire or other materials described herein) on medical devices that allow for improved control over tissue penetration depth with decreased risk of damaged cardiac tissue and related complications such as pericardial effusion, while still providing adequate engagement of the medical device. Additionally, it would be desirable to incorporate multi-legged stabilizing members, stabilizing members having singular or multi-hooked and/or barbed engagement portions, and stabilizing members further having complex hook geometries and/or complex barb configurations.

The medical devices of the present disclosure, which include stabilizing members having improved penetration depth control, extension control, and/or engagement control via optimized shaping, geometries (e.g., hooked engagement portions, and/or barbed engagement portions), materials, construction, and/or attachment serve to avoid various potential disadvantages of known or conventional medical devices.

Accordingly, the medical devices of the present disclosure enable suitable tissue engagement and dislodgement prevention of the medical devices while reducing damage to the cardiac tissue and the risk of related complications such as pericardial effusion by providing control of penetration depth into the tissue with stabilizing members having engagement portions including hooked and/or barbed designs.

In some embodiments disclosed herein, stabilizing members (e.g., formed from wire or other materials described herein) are designed and optimized to control tissue penetration depth by controlling and/or limiting stabilizing member extension from the body of the device (e.g., such as a lobe and/or disc of the device body). In some embodiments disclosed herein, stabilizing members for the medical devices are designed and optimized to reduce the risk of perforation of the left atrial appendage. In some embodiments disclosed herein, stabilizing members are made from nitinol wire, laser cut nitinol, and/or other materials described herein, as an alternative to or in conjunction with conventional wire stabilizing members. Advantages of the embodiments described herein include enabling stabilizing member penetration depth control, and stabilizing member designs having more than one engagement portion (e.g., hooks and/or barbs) on a stabilizing member and/or complex engagement portion geometries.

The disclosed embodiments may lead to more consistent and improved patient outcomes. It is contemplated, however, that the described features and methods of the present disclosure as described herein may be incorporated into any number of systems as would be appreciated by one of ordinary skill in the art based on the disclosure herein.

It is understood that the use of the term “target site” is not meant to be limiting, as the medical device may be configured to treat any target site, such as an abnormality, a vessel, an organ, an opening, a chamber, a channel, a hole, a cavity, or the like, located anywhere in the body. The term “vascular abnormality,” as used herein is not meant to be limiting, as the medical device may be configured to bridge or otherwise support a variety of vascular abnormalities. For example, the vascular abnormality could be any abnormality that affects the shape of the native lumen, such as an LAA, an atrial septal defect, a lesion, a vessel dissection, or a tumor. Embodiments of the medical device may be useful, for example, for occluding an ASD, LAA, PDA, PFO, or VSD, as noted above. Furthermore, the term “lumen” is also not meant to be limiting, as the vascular abnormality may reside in a variety of locations within the vasculature, such as a vessel, an artery, a vein, a passageway, an organ, a cavity, or the like. As used herein, the term “proximal” refers to a part of the medical device or the delivery device that is closest to the operator, and the term “distal” refers to a part of the medical device or the delivery device that is farther from the operator at any given time as the medical device is being delivered through the delivery device.

The medical device may include one or more layers of occlusive material, wherein each layer may comprise any material that is configured to substantially preclude or occlude the flow of blood so as to facilitate thrombosis. As used herein, “substantially preclude or occlude flow” shall mean, functionally, that blood flow may occur for a short time, but that the body's clotting mechanism or protein or other body deposits on the occlusive material results in occlusion or flow stoppage after this initial time period. The medical device may include a device body (e.g., at least one disc and/or lobe), wherein at least a portion of the device body is formed from a shape memory material. One particular shape memory material that may be used is nitinol. Nitinol alloys are highly elastic and are said to be “superelastic,” or “pseudoelastic.” This elasticity may allow medical device to be resilient and return to a preset, expanded configuration for deployment following passage in a distorted form through a delivery system (e.g., a delivery catheter). Further examples of materials and manufacturing methods for medical devices with shape memory properties are provided in U.S. Pat. No. 8,777,974, titled “Multi-layer Braided Structures for Occluding Vascular Defects” and filed on Jun. 21, 2007, which is incorporated by reference herein in its entirety. It is also understood that the medical device may be formed from various materials other than nitinol that have elastic properties, such as stainless steel, trade named alloys such as Elgiloy®, or Hastalloy, Phynox®, MP35N, CoCrMo alloys, metal, polymers, or a mixture of metal(s) and polymer(s). Suitable polymers may include PET (Dacron™), polyester, polypropylene, polyethylene, HDPE, polyurethane, silicone, PTFE, polyolefins and ePTFE. The shape memory material may comprise a braided mesh fabric. In exemplary embodiments, device body (e.g., at least one disc and/or lobe) is formed from a braided shaped-memory material (e.g., a braided nitinol fabric or other mesh material, such as PE, PET, Si, PLLA, PLGA, PlA, PLLA-PLC, etc.), to provide an occlusive effect. Moreover, a braided mesh fabric material enables the medical device to be selectively transitioned from an expanded configuration to a collapsed configuration for delivery (e.g., through delivery catheter), and return to the expanded configuration upon deployment at the target site.

The present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

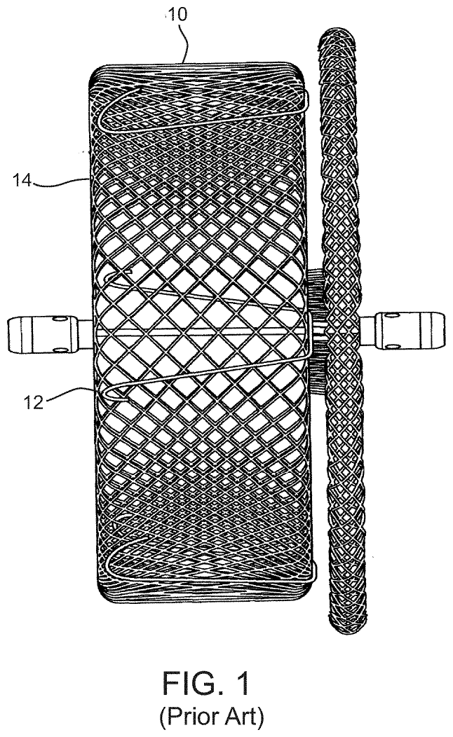

In at least some conventional or known medical devices, such as a medical device 10 shown in FIG. 1, conventional wires 12 extend from a device body 14 of medical device 10. These conventional wires 12 are configured to retain medical device 10 at a desired target site within a human body, and prevent medical device 10 from being dislodged from the target site after deployment of medical device 10. Any type of device anchors or stabilizing members (such as wires, hooks, etc.) should be long enough to engage surrounding tissue and provide stability for the device. The stabilizing members described herein are configured to circumvent problems that may be associated with decreased control with respect to tissue penetration depth. For instance, when medical device 10 is deployed to a desired target site within a human body such as for occlusion of the left atrial appendage, the stabilizing members described below effectively improve tissue penetration control, and prevent cardiac tissue penetration that is too deep, thereby avoiding cardiac tissue damage, pericardial effusion, and/or other complications.

Penetration Depth Control

As described herein, some embodiments serve to address penetration depth control of the stabilizing members 200 of target site and surrounding tissue, such as LAA tissue. In some embodiments, at least one of extension control, and/or engagement control is additionally and advantageously imparted by the design of stabilizing member 200. It is therefore contemplated that the various features described in the following penetration depth control embodiments may be used in combination with one or more other penetration depth control features and/or in combination with one or more extension control, and/or engagement control features described elsewhere herein. Consequently, figures and embodiments showing a single engagement portion and/or a single backing portion with various features are also representative of embodiments having two or more engagement portions (e.g., multi-hooked stabilizing members) and/or embodiments having two or more backing portions (e.g., multi-legged stabilizing members) with the exemplified feature(s). In a similar manner, multi-engagement portion and/or multi-backing portion embodiments exemplifying various features are also representative of single engagement portion and/or single backing portion embodiments with the exemplified feature(s).

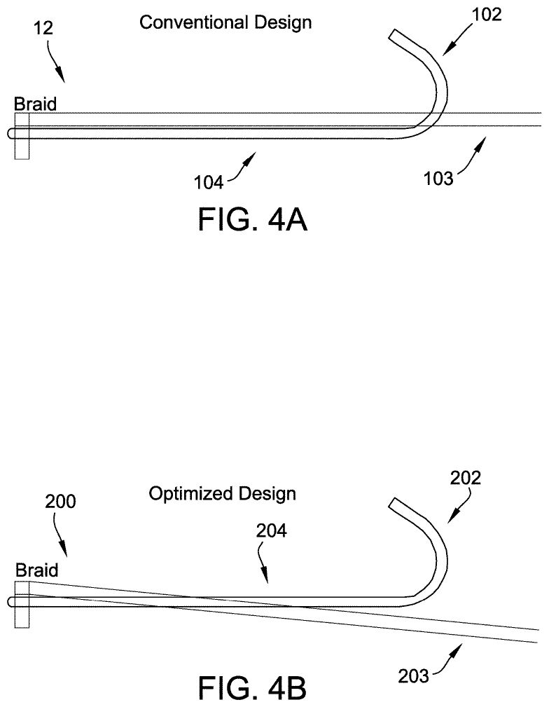

Turning now to FIG. 4A, a conventional design is shown, in which a conventional wire 12 is situated generally in-line with (or generally parallel to) braid 103 (such as a braided outer layer covering device body 14 of medical device 10, as shown in FIG. 1). A majority of a hooked engagement portion 102 of conventional wire 12 is located outside of braid 103, while stabilizing leg 104 remains inside of braid 103. In contrast, FIG. 4B depicts an optimized design in which a stabilizing member 200 (e.g., formed from wire or other material described herein) is attached to a medical device (not shown) in such a way that an engagement portion 202 of stabilizing member 200 emerges from or exits braid 203 sooner than the conventional design, thereby being placed more fully external to braid 203, rather than internal to braid 203. In this embodiment, an entirety of engagement portion 202 of stabilizing member 200, as well as a portion of backing portion 204, is placed external to (e.g., outside of) braid 203. The embodiment shown in FIG. 4B provides a more rigid support for stabilizing member 200 than the conventional design embodiment shown in FIG. 4A. With extra support, engagement portion(s) 202 may be cut shorter in some embodiments, thus reducing overall penetration depth of the engagement portion of the medical device.

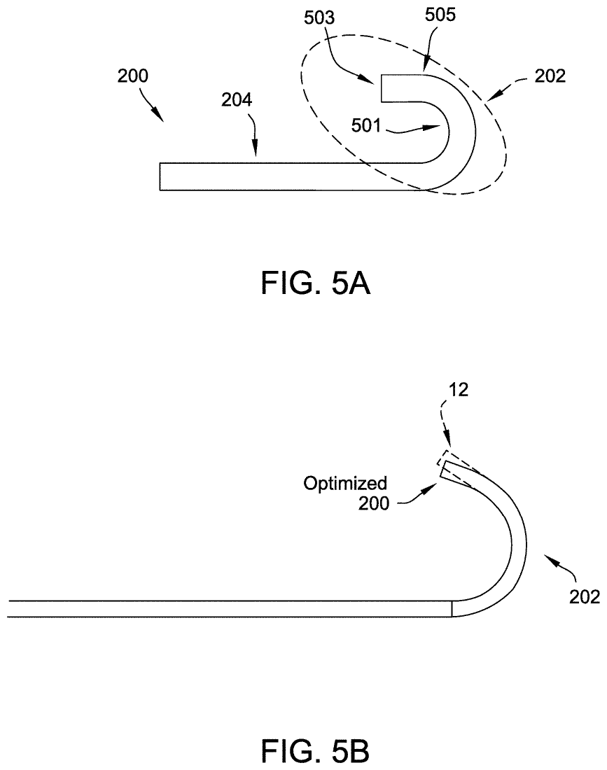

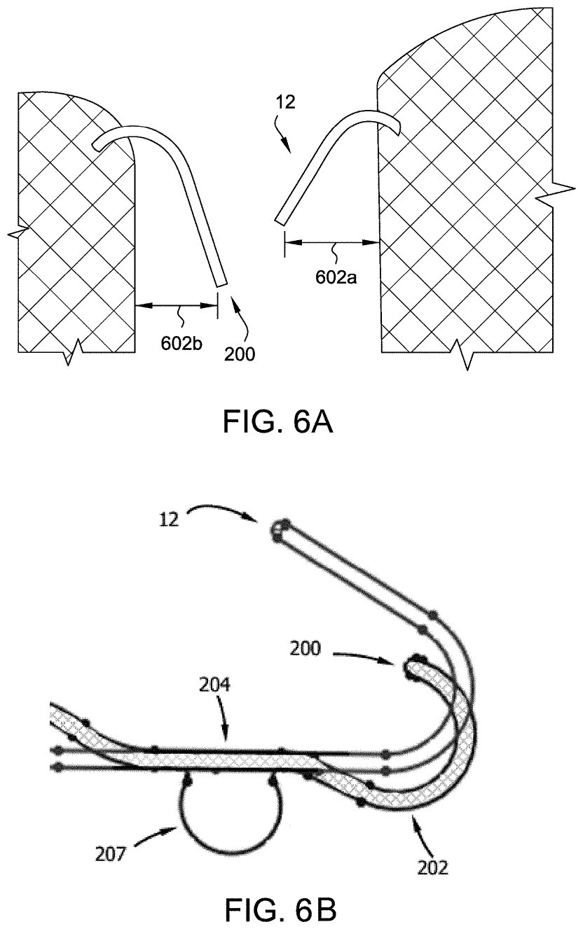

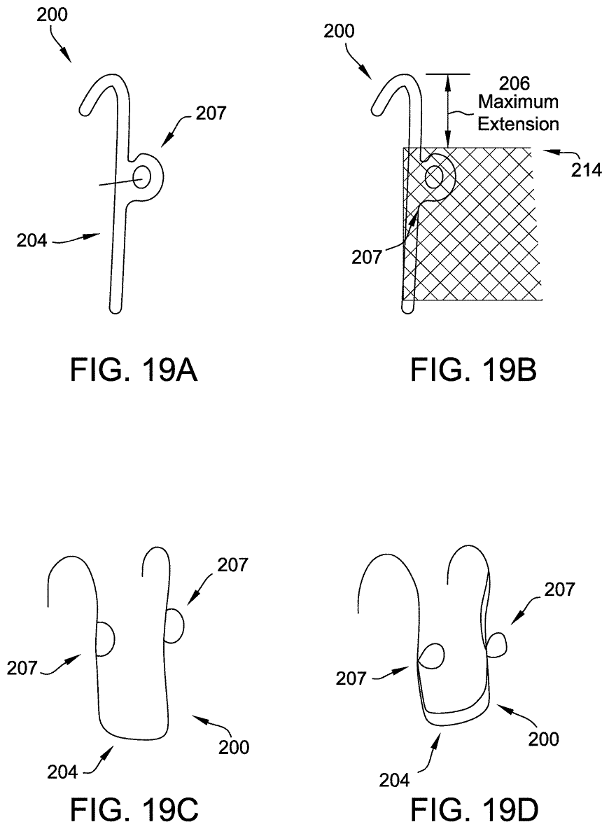

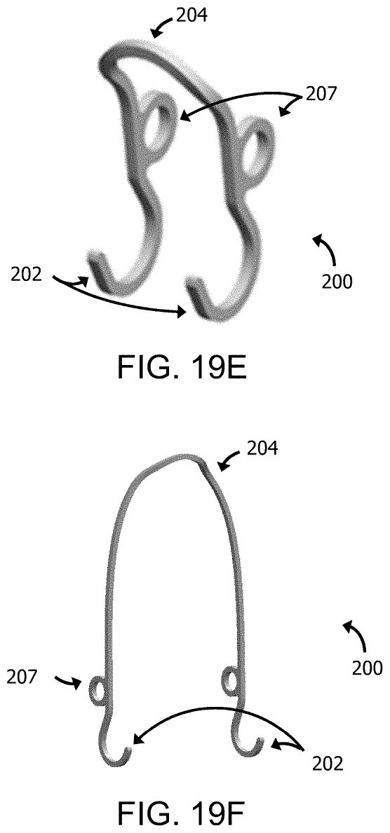

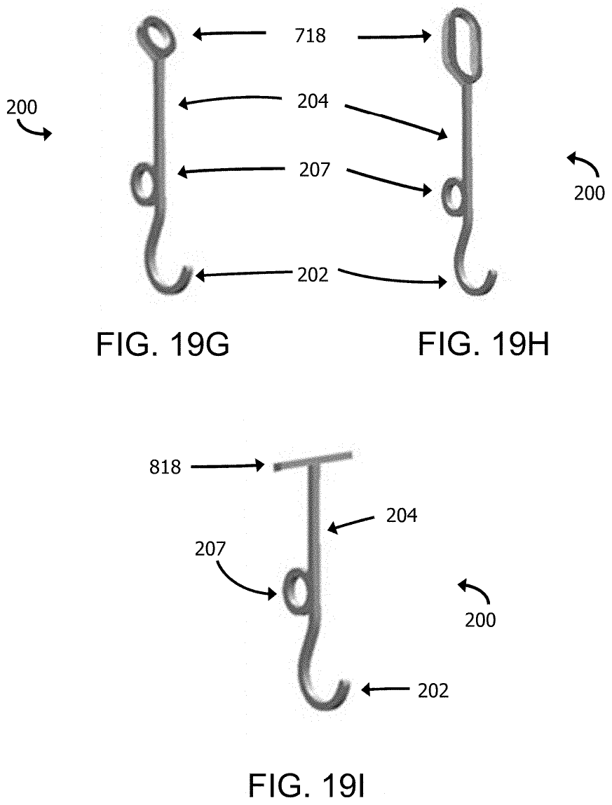

FIG. 5A, FIG. 5B, FIG. 6A, and FIG. 6B depict stabilizing members 200 (formed from wire or other material described herein) including a relatively shallower hook angle when compared to conventional designs. In some embodiments where engagement portion 202 of stabilizing member 200 is hooked or curved, engagement portion 202 may include a proximal curved portion 501 adjacent to a distal linear portion 503 (see FIG. 5A). In exemplary embodiments, an angle formed when distal linear portion 503 of engagement portion 202 exits from the adjacent proximal curved portion 501 (e.g., at a point 505 tangential to the arc of proximal curved portion 501, as shown in FIG. 5A) is configured to be less than a corresponding angle of the conventional wire 12 design (see FIG. 5B, FIG. 6A, and FIG. 6B). In exemplary embodiments, distal linear portion 503 may range from about a 5 L tangent length (0.005 inches) to a 20 L tangent length (0.020 inches). The hook angle affects a depth 602 measured between the distal end of engagement portion 202 (and therefore also distal end stabilizing member 200) and the device body (e.g., a lobe of the device body) when the device is in the deployed configuration. As shown in FIG. 6A and FIG. 6B, an optimized depth 602b is smaller/shorter than a conventional depth 602a due to the shallower angle of the distal end of engagement portion 202 relative to the proximally-adjacent curved portion of engagement portion 202. A conventional depth 602a is typically about 0.3 inches. In some embodiments disclosed herein, depth 602b may range from about 0.01 inches to about 0.15 inches, or from about 0.02 to about 0.10 inches, or about 0.05 inches. In these embodiments, a smaller/shallower hook angle reduces the risk that engagement portion 202 of stabilizing member 200 penetrates deep into the LAA wall, and instead it would penetrate to a shallower, more controllable depth. In some embodiments, a combination of hook angle and hook radius (see FIG. 6B) is implemented in order to achieve effective attachment (e.g., by achieving, at least in part, a desired depth 602 as shown in FIG. 6A) of the device when deployed at the target site. FIG. 6B additionally illustrates an exemplary stabilizing member 200 having an eyelet 207 for extension control (as described in greater detail below with respect to FIGS. 19A-D).

Moreover, a recess angle (see FIG. 6B where engagement portion 202 no longer extends straight from backing portion 204) aids in improved re-capturability of the medical device by allowing the delivery sheath to bend the engagement portion 202 (e.g., hook) more easily back into the device body (e.g., into a lobe of the device body), thus reducing user force to recapture the medical device. Stabilizing members with recess angles are also shown in FIGS. 19E-I, below. In some embodiments, a rounded edge on the tip of engagement portions 202 (e.g., hooks) allow for full recapture back into the sheath without causing damage to the sheath itself. For example, in these embodiments the rounded edge may be formed by rounded laser cut designs further processed with electropolishing to round the tip, or may be formed from a round wire stabilizing member 200 with a welded tip to round the edges.

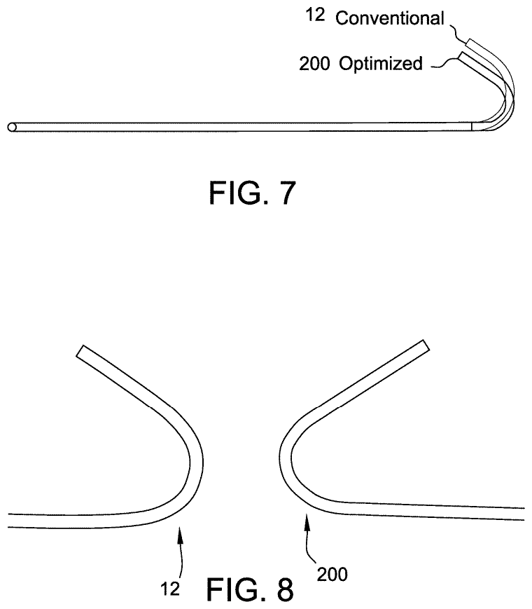

FIGS. 7 and 8 depict stabilizing members 200 (formed from wire or other material described herein) including a relatively smaller bend radius or arc. In some embodiments where engagement portion 202 is hooked/curved (see FIG. 5A), a bend radius or arc of proximal curved portion 501 of engagement portion 202 of stabilizing member 200 is formed to be smaller than that of the conventional wire 12 (see FIG. 7 and FIG. 8). In the exemplary embodiments, a smaller bend radius imparts increased stiffness at the bend of engagement portion 202; thereby lowering the chance that engagement portion 202 is bent further into the LAA wall than is necessary. As discussed above, a smaller bend radius or smaller arc of curved portion 501 may be used (either alone or in conjunction with hook angle described above) to achieve a desired optimized depth 602b between the distal end of stabilizing member 200 and the body (e.g., a lobe of the device body) of the deployed device. In some embodiments disclosed herein, depth 602b may range from about 0.01 inches to about 0.15 inches, or from about 0.02 to about 0.10 inches, or about 0.05 inches. Depending on the embodiment, optimized depth 602b may be achieved by forming a smaller bend radius based on a suitable inner and/or outer radius of curved portion 501.

As described herein, some additional embodiments serve to address penetration depth control of stabilizing members 200, to reduce or eliminate a risk of stabilizing members 200 extending too far into surrounding tissue of a target site. For instance, utilizing laser cut nitinol as an alternative to nitinol wire for stabilizing member 200 construction enables formation of multiple engagement portions 202 on a stabilizing member 200 and enables formation of complex engagement portion 202 geometries, each and both of which contribute to overall control of penetration depth.

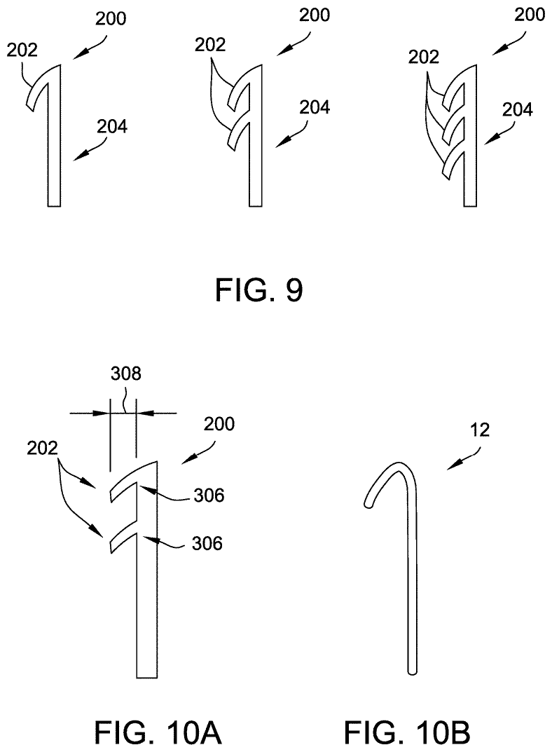

Turning now to FIG. 9, an exemplary embodiment of multi-hooked engagement portions 202 of a stabilizing member 200 is shown in accordance with the present disclosure. Stabilizing member(s) 200 are coupled to a body of the medical device. In some exemplary embodiments, engagement portion(s) 202 extend from a backing portion 204 (e.g., a leg) of stabilizing member 200, and backing portion 204 is coupled directly to a body of the medical device. Specifically, each engagement portion 202 extends from an outward face of the backing portion 204. Engagement portion 202 is configured to extend radially outwardly from a device body of a medical device (e.g., an occluder). In an exemplary embodiment, there may be one, two, three, or more engagement portions (e.g., hooks) 202 on a single stabilizing member 200, thus providing increased levels of anchoring for the medical device as desired.

In an exemplary embodiment, stabilizing members 200 are laser cut from a suitable material form, such as a tube or flat sheet of the material. Materials may include shape memory alloys and polymers such as shape memory polymers and bio-absorbable polymers. One particular shape memory alloy that may be used is nitinol. In another exemplary embodiment, stabilizing members 200 are injection molded using a polymer. Suitable polymers for laser cutting and injection molding also include thermoplastics such as nylon and/or Pebax™, shape memory polymers, and bio-absorbable polymers. Injection-molded stabilizing members 200 may be attached to a device body of a medical device by further injection molding, by sewing, or combinations thereof, depending on the embodiment. Shape memory alloy stabilizing members 200 may also be attached using injection molding and/or sewing to a medical device body.

One advantage of creating or forming stabilizing members 200 (such as hooked stabilizing members) by laser cutting or injection molding includes enabling formation of more than one hook (or other engagement portion) on a single stabilizing member (as shown in FIG. 9). In some embodiments, shorter engagement portions 202 (e.g., shorter hooks) are more desirable to limit pericardial effusion, however, more hooks may be necessary in order to achieve an adequate level of anchoring of the medical device to the tissue. Multiple engagement portions 202 on one stabilizing member 200 may enable adequate device-to-tissue anchoring without increasing the amount of attachment/coupling required (e.g., by sewing and/or injection molding, as described herein) in order to fasten and secure stabilizing members 200 to the device body.

FIG. 10A shows an exemplary embodiment of tissue penetration depth limit of a stabilizing member 200 in accordance with the present disclosure. Another advantage of creating stabilizing members 200 by laser cutting or injection molding includes enabling penetration depth control (as shown in FIG. 10A). With a laser cut profile of stabilizing member 200, relatively tighter bends or corners can be created, thus creating a discernable boundary (e.g., a penetration stop) between engagement portion 202 and backing portion 204. These tighter corners provide depth control for tissue penetration, and controlling the depth of tissue penetration may reduce the likelihood of pericardial effusion. However, as described above and shown in FIG. 10B, conventional wire 12 of some known medical devices has a gentle curve, which lacks a discernable boundary between a backing-type portion of the wire and an engagement-type portion of the conventional wire 12, and therefore, alone, provides little limit to tissue penetration depth of the conventional wire 12. In an example embodiment of the present disclosure, a hooked engagement portion 202 of stabilizing member 200 includes penetration stops 306 defined by the tight corners created with the laser cut profile of stabilizing member 200. In the embodiment shown in FIG. 10A, at least one penetration stop 306 is provided at each hooked engagement portion 202. Consequently, a stabilizing member 200 has a penetration limit 308 as defined by the length of the engagement portion 202 that extends from the backing portion 204. In exemplary embodiments, penetration limit 308 is up to about 2 mm, up to about 1.5 mm, or up to about 1 mm.

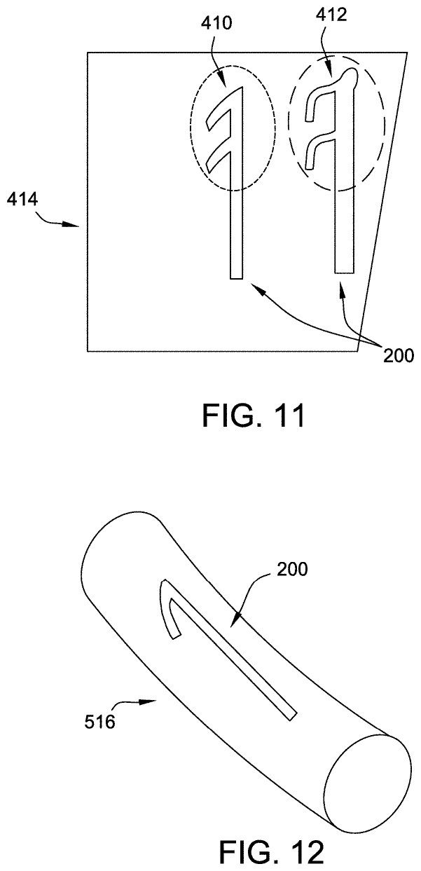

FIG. 11 shows an exemplary embodiment of a complex-geometry hooked engagement portion of a stabilizing member in accordance with the present disclosure. Yet another advantage of creating the stabilizing members 200 (such as hooked stabilizing members) by laser cutting or injection molding includes enabling complex hook geometries, as shown in FIG. 11. Complex engagement portion geometries (such as hook geometries) can be created or formed using laser cut profiles that may be more difficult or impossible with a wire design. More complex hook geometries may further facilitate resolving pericardial effusion. For example, a simple, predominantly linear hook geometry 410 is generally shown within a dotted circle in FIG. 11, in which the hooked engagement portions extend out from the backing portion in a predominantly straight direction with a predominantly straight geometry. A more complex geometry 412 is generally shown within a dashed circle in FIG. 11, in which the hooked engagement portions extend out from the backing portion in a combination of straight segments as well as curved segments. In some embodiments, stabilizing members 200 are laser cut from a flat sheet 414 form of alloy, such as nitinol. This enables complex hook geometries (such as the hook geometry 412), along with all the other advantages of laser cut designs as described herein. In some embodiments, a single stabilizing member 200 having a plurality of hooks may have varying hook geometries.

FIG. 12 shows an exemplary embodiment of a stabilizing member 200 that is laser cut from a tubular material 516 in accordance with the present disclosure. In an exemplary embodiment, stabilizing members 200 having stabilizing hook engagement portions (e.g., engagement portions 202) are laser cut from a nitinol tube 516. One advantage of using a tube form 516 is that the curvature of the tube 516 imparts a curvature to the engagement portion 202.

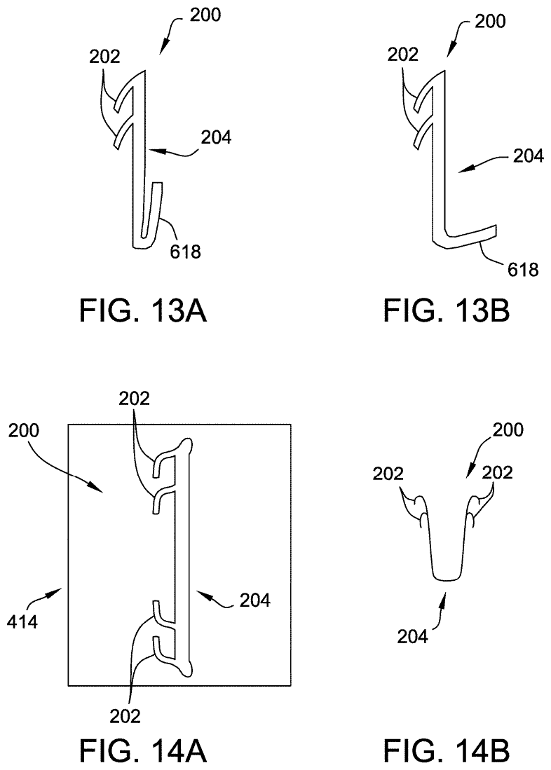

Turning now to FIGS. 13A-B and 14A-B, embodiments of stabilizing members 200 are shown, including various features to address rotation of the engagement portion 202 of a stabilizing member 200. When stabilizing member 200 is coupled to the body of the medical device, rotation may occur such that engagement portion 202 (e.g., one or more hooks) is not oriented to interact with the tissue. Therefore in some embodiments, it is desirable to prevent rotation of engagement portion 202 away from the tissue that engagement portion 202 is intended to engage when the medical device is deployed.

One option is to further include a rotation-prevention feature or rotation restraint such as tail 618 on stabilizing member 200, wherein tail 618 is coupled to an interior of the device body (FIG. 13A-B), thus preventing rotation of stabilizing member 200 and, therefore, of engagement portion 202. In the exemplary embodiments, tail 618 extends from a bottom of backing portion 204. Tail 618 may extend generally parallel to backing portion 204 (e.g., to form a “U” shape with backing portion 204, FIG. 13A). In some such embodiments, tail 618 is bent to form a “U” shape with backing portion 204 before attachment to the device body. Alternatively, tail 618 may extend generally perpendicularly from backing portion 204 (FIG. 13B). Embodiments having a rotation restraint similar to tail 618 (e.g., a base loop or T-bar) are described herein below with respect to FIGS. 19G-I.

Another option is forming (e.g., laser cutting) stabilizing members 200 in pairs by connecting a base of each leg (e.g., backing portion 204) by a section of material (FIG. 14A), and manipulating the stabilizing members 200 to form a “U” shaped backing portion 204, such as by heat-setting (FIG. 14B). Section(s) of material connecting two or more backing portions 204 may be straight and/or curved as suitable to form multi-legged stabilizing members 200. Stabilizing member 200 is coupled to the device body in the “U” shape, thereby preventing rotation of stabilizing member 200 and, therefore, of engagement portions 202. In some embodiments, laser cutting stabilizing members 200 (e.g., having hooked engagement portions) from a flat sheet of nitinol or from a nitinol tube, and forming the cut stabilizing members 200 in a geometry similar to a “U” shape of conventional wires 12 (see FIG. 3), such as shown in FIG. 14B, allows for more complex geometries than would otherwise be achievable with formed nitinol wire. In some embodiments, stabilizing members 200 have multiple points of contact, i.e., multiple engagement portions 202 per member 200. These multiple points of contact allow for both force dispersal and depth control, such as in cases of LAA penetration at a single location.

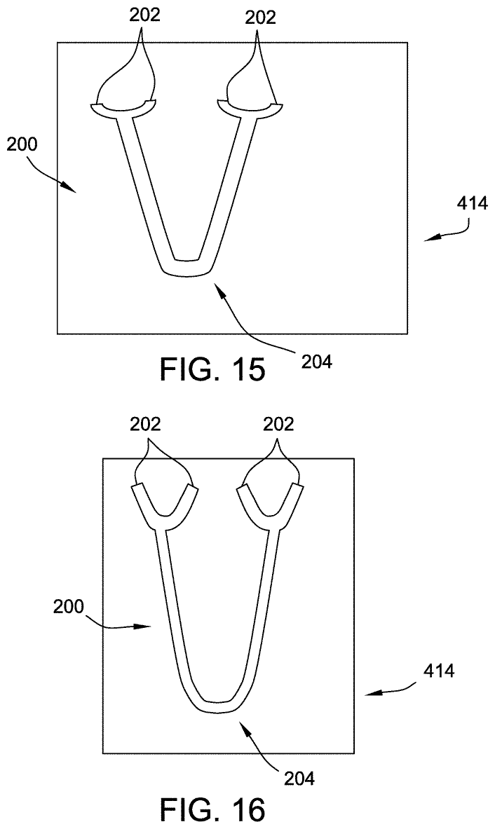

As shown in FIG. 15-17, stabilizing members 200 are laser cut from flat sheet nitinol 414, enabling complex engagement portion 202 (e.g., hook) geometries, along with other advantages of laser cut designs, including those described elsewhere herein. In some embodiments, stabilizing members 200 are alternatively cut from a nitinol tube 516 (not shown) to impart a curvature from tube 516 to the engagement portion(s) 202, backing portion 204, or both.

FIG. 15 and FIG. 16 depict embodiments that address the dispersal of forces experienced by the engagement portion 202 (e.g., the hook) of a stabilizing member 200. In conventional wire designs, there is a total force F pulling the device towards the atrium of the heart. If each tip of all the conventional wires are contacting the pectinate muscle of the LAA, that force is dispersed evenly (e.g., F/20, for medical device with 20 conventional wires). By utilizing stabilizing member embodiments described herein alternatively to conventional wires, and by splitting the ends of each engagement portion 202 and considering all tips of the engagement portions 202 make contact, the force is cut in half (F/40). In some embodiments, a wider hook concept (such as shown in FIG. 15) prevents hooks from slipping between pectinate muscle to the thin wall areas of the LAA.

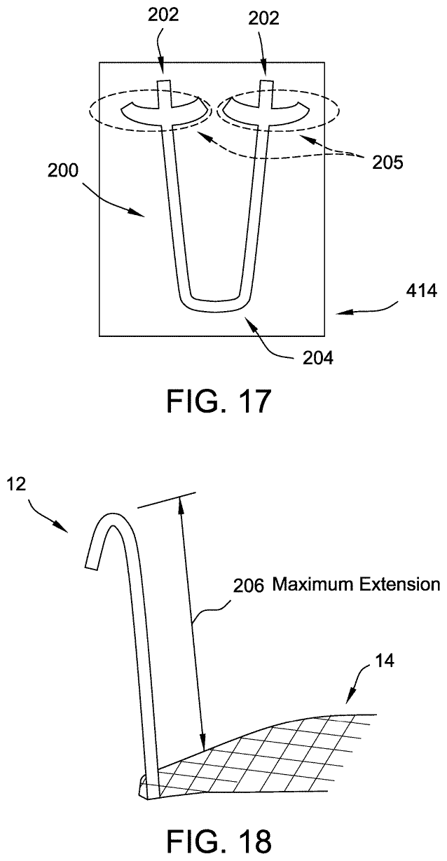

FIG. 17 shows a design similar to conventional wires with added penetration stops such as guards 205 on either side, extending from backing portion 204 for depth control. Guard 205 serves as a type of penetration stop (such as penetration stop 306 shown in FIG. 10A). In this embodiment, when penetration of the tissue occurs upon deployment, guards 205 aid in preventing engagement portions 202 (e.g., hooks) from engaging any further through the LAA wall, resulting in surrounding cardiac structures, such as the circumflex artery, being protected against ancillary penetration.

In an exemplary embodiment, stabilizing member 200 is formed from a singular or continuous piece of material by laser cutting an alloy form (such as a tube or flat sheet of nitinol) or by injection molding both engagement portion 202 and backing portion 204 using a single mold. In a further exemplary embodiment, engagement portion 202 and backing portion 204 are formed separately using the same shape memory material (such as an alloy or polymer) and subsequently coupled together. In yet a further exemplary embodiment, engagement portion 202 and backing portion 204 are formed from different materials (e.g., different alloys, different polymers, or both) and subsequently coupled together.

Extension Control

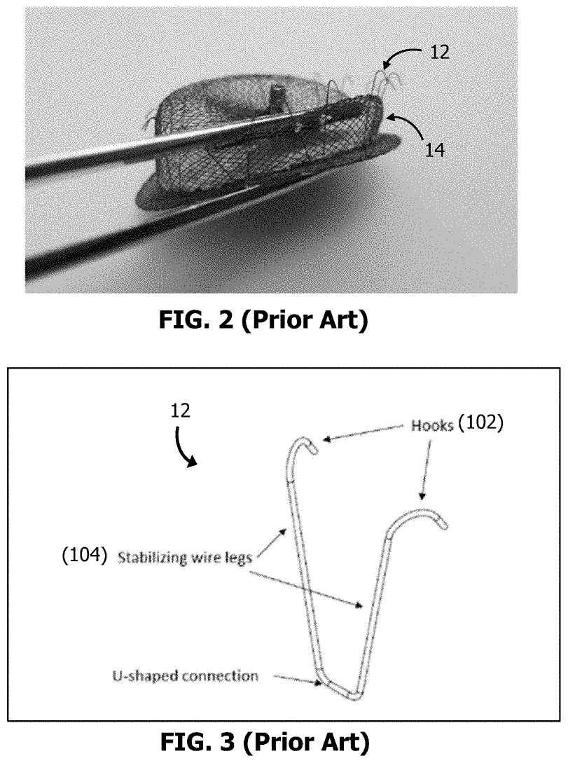

As described herein, some embodiments address extension control of the stabilizing members 200 too far beyond the device body 14 (e.g., from a lobe and/or disc of the device) of medical device 10 (as illustrated in FIG. 2). In some embodiments, at least one of penetration depth control, and/or engagement control is additionally and advantageously imparted by the design of stabilizing member 200. It is therefore contemplated that the various features described in the following extension control embodiments may be used in combination with one or more other extension control features and/or in combination with one or more penetration depth control, and/or engagement control features described elsewhere herein. Consequently, figures and embodiments showing a single engagement portion and/or a single backing portion with various features are also representative of embodiments having two or more engagement portions (e.g., multi-hooked stabilizing members) and/or embodiments having two or more backing portions (e.g., multi-legged stabilizing members) with the exemplified feature(s). In a similar manner, multi-engagement portion and/or multi-backing portion embodiments exemplifying various features are also representative of single engagement portion and/or single backing portion embodiments with the exemplified feature(s). A representation of a conventional wire 12 design and a maximum amount of extension 206 from a compressed device body 14 is shown in FIG. 18.

Turning now to FIGS. 19A-I, stabilizing member 200 includes at least one eyelet 207 (e.g., formed from wire or other material described herein, or formed from a laser cut design as described herein). FIG. 19A depicts a stabilizing member 200 with eyelet 207, and in a deployed configuration (FIG. 19B) having a maximum extension from the braid of device body. In this embodiment, eyelet 207 is located at a desired point along backing portion 204, depending on a desired amount of maximum extension 206 (i.e., a maximum extension length of the stabilizing member from the device body). Stabilizing member 200 is attached to device body 214 (e.g., a braided nitinol lobe or disc having braid 203 described above) at eyelet 207, (e.g., by threading material through eyelet 207 such that stabilizing member 200 is sewn to device body 214 at eyelet 207). Depending on the embodiment, eyelet 207 can be at any location along backing portion 204. By constraining device body 214 to eyelet 207 (e.g., by sewing eyelet 207 to device body 214 with at least one stitch), backing portion 204 is restricted from movement with respect to device body 214, keeping the majority of the stabilizing member 200 within device body 214 (e.g., within the braid of a braided outer layer). Eyelet 207 feature exemplified in the embodiments of FIG. 19C and FIG. 19D also ensures consistent protrusion of engagement portion 202 and engagement with tissue upon deployment of the device. In these embodiments, eyelet 207 location effectively prevents tilting of engagement portion 202 back into the braid (e.g., for shorter length engagement portion embodiments), and also ensures consistent tissue contact. Consequently, in addition to extension control, eyelet 207 feature may also contribute to penetration depth control as described above. In some embodiments, such as shown in FIG. 19C and FIG. 19D, stabilizing member 200 is formed from round wire and also has at least one eyelet 207 formed from the wire at a desired point along backing portion 204. In other embodiments, such as shown in FIG. 19E and FIG. 19F, stabilizing member 200 may be formed either by wire or by laser cut design and include at least one eyelet 207.

FIGS. 19G-I illustrate embodiments of stabilizing members 200 having rotation restraints such as a loop or T-bar to prevent rotation of engagement portion 202. FIGS. 19G and 19H show a base loop 718 located at a base of backing portion 204 Base loop 718 may be more circular (FIG. 19G) or more oblong (FIG. 19H) depending upon the embodiment to accommodate the length change of the braid when the device transitions between collapsed and expanded configurations. FIG. 19I shows a T-bar 818 located at a base of backing portion 204 for preventing rotation of engagement portion 202. Base loop 718 and T-bar 818 rotation restraints may be similar to tail 618 rotation restraint, see FIGS. 13A and 13B. These rotation restraint embodiments are particularly configured for single-legged stabilizing member embodiments where engagement portion(s) 202 are only located at one end of backing portion 204. However, in alternatives embodiments, rotation restraints described herein may be additionally present at a base of “U” shaped backing portion 204 of embodiments such as those shown in FIGS. 14A and 14B. It is further contemplated that stabilizing members with rotation restraints may include one or more engagement portion 202 positioned at an opposite end of backing portion 204 from the rotation restraint feature (e.g., FIG. 13A). In embodiments where eyelet 207 is present, base loop 718 or T-bar 818 rotation restraint further prevents eyelet 207 from twisting such that engagement portion 202 properly maintains an outward-facing direction relative to the device. Depending upon the embodiment, base loop 718 or T-bar 818 rotation restraints may be positioned either interior or exterior to a braided outer layer of the device, while eyelet 207 will be positioned interior to the braided outer layer for extension control. In these embodiments, stabilizing member 200 may be attached to device body 214 at the rotation restraint (i.e., at base loop 718 or T-bar 818), or may alternatively be attached at another location of stabilizing member 200.

In the embodiments of FIGS. 19E-I, engagement portion 202 no longer extends linearly straight from backing portion 204 but rather curves to form a recess angle (see also FIG. 6B) to improve re-capturability of the medical device by allowing a delivery sheath to bend engagement portion 202 more easily back into the device body (e.g., into a lobe of the device body), thereby reducing user force to recapture the medical device.

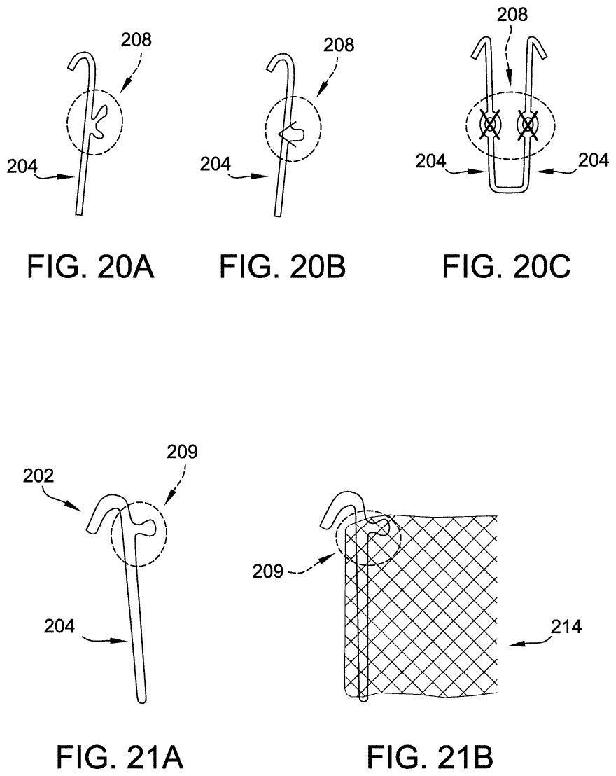

In some embodiments, as depicted in FIG. 20A, FIG. 20B, and FIG. 20C, any attachable feature 208 that would, upon attachment to device body 214, restrict backing portion 204 from movement relative to device body 214 may be used as an alternative to eyelet 207, such as any suitable protuberance, node, knob, projection, or ledge, etc. that is attachable to device body 214.

In some embodiments, an arresting feature 209, such as depicted in FIG. 21A and FIG. 21B, is located near the distal aspect of backing portion 204 (i.e., nearer to engagement portion 202). Similar to attachable feature 208, arresting feature 209 can be any suitable protuberance, node, knob, projection, or ledge, etc. In contrast to attachable feature 208, arresting feature 209 would not require attachment to device body 14. That is, arresting feature 109 would be located on the side of the stabilizing member that is interior to device body 214 (e.g., interior to a braided outer layer), and would be large enough that it does not easily pass through the cells or braid of device body 214 (FIG. 21B). Therefore, engagement portion 202 (such as hooks or barbs described herein elsewhere) can extend out of device body 214 (such as extending out of a lobe of the device body and/or extending out of a disc of the device body) only until arresting feature 209 is “arrested” or “caught” against an interior surface of device body 214.

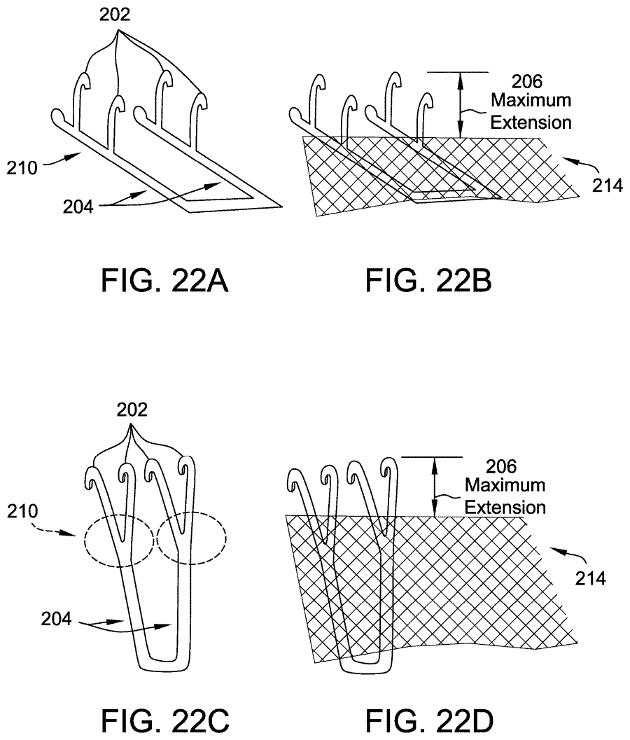

FIG. 22A, FIG. 22B, FIG. 22C, and FIG. 22D show embodiments in which multiple engagement portions 202 (e.g., hooks) extend from each backing portion 204. Each engagement portion 202 is sized, positioned, and oriented to exit from a different opening of device body 214 (e.g., a different cell of the braided outer layer). Locations at which each engagement portion 202 extends from its respective backing portion 204 form bifurcations 210 that are configured to engage with/against the interior of device body 214 to prevent further radially-outward movement. Therefore, each engagement portion 202 cannot extend from device body 214 beyond the bifurcations 210 in backing portion 204, and the maximum extension 206 is limited.

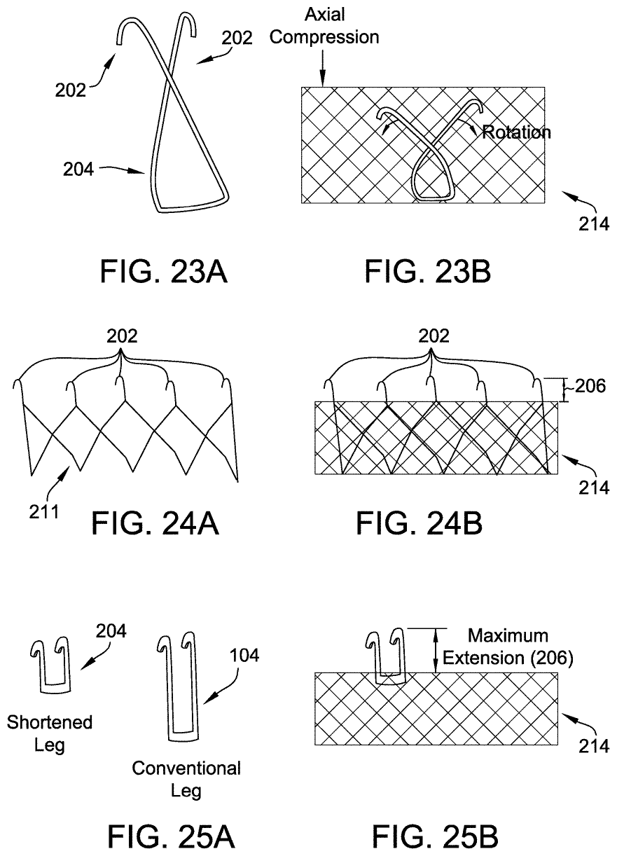

In the embodiment shown in FIG. 23A, backing portions 204 of stabilizing member 200 cross over one another. Therefore, when device body 214 is compressed axially (FIG. 23B), backing portions 204 tend to rotate within device body 14 openings/cells rather than slide out of or extend excessively beyond device body 214.

FIG. 24A illustrates an embodiment in which multiple stabilizing members are replaced with a single stent-like structure 211 with engagement portions 202 (e.g., hooks) at the distal aspect to engage target site tissue. In some embodiments, the entire stent-like structure 211 is placed into or onto device body 214 and suitably attached in place (such as by sewing and/or injection molding). The presence of the diamond-shaped cells of the stent-like structure 211 prevents extension of engagement portions 202 beyond where they attach to the cells (FIG. 24B).

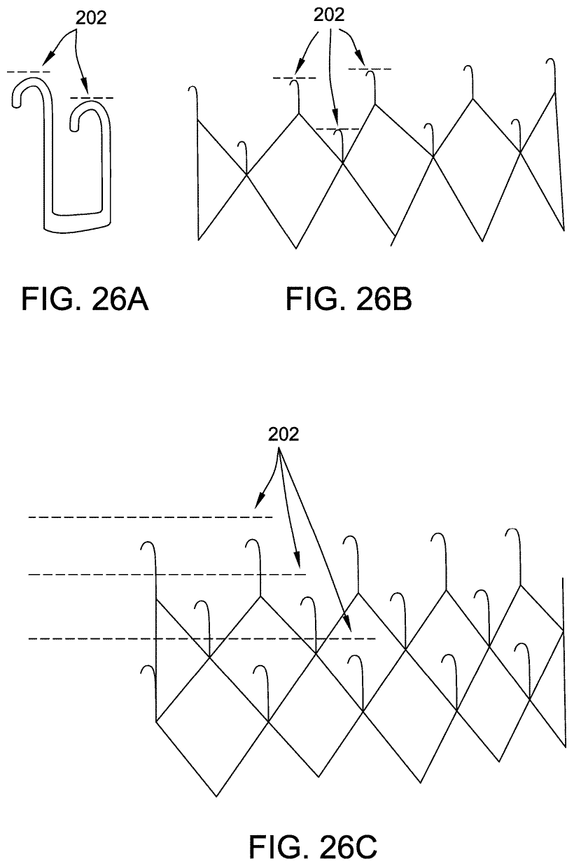

In the embodiment shown in FIG. 25A, the length of backing portion 204 is shortened, relative to conventional hooked wire designs. Conventional wire legs, such as legs 104 shown in FIG. 3, typically range from about 0.3 inches to 0.4 inches for larger devices and from about 0.2 inches to about 0.3 inches for smaller devices. In some embodiments disclosed herein, a length of backing portion 204 for a generally larger medical device (e.g., a device generally sized for an adult or a device generally sized for larger target sites) ranges from about 0.03 inches to about 0.2 inches, or about 0.04 inches to about 0.15 inches, or about 0.05 inches to about 0.10 inches. In these embodiments, the leg height of backing portion 204 for a generally larger medical device allows the engagement portions (e.g., hooks) to be radially spaced. In other embodiments disclosed herein, a length of backing portion 204 for a generally smaller medical device (e.g., a device generally sized for a child or a device generally sized for smaller target sites) ranges from about 0.01 inches to about 0.15 inches, or about 0.02 inches to about 0.10 inches, or about 0.03 inches to about 0.05 inches. In these embodiments, the leg height of backing portion 204 for a generally smaller medical device allows for multiple rows of engagement portions, as shown herein below (e.g., FIG. 26B and FIG. 26C). According to these aspects, backing portion 204 is still permitted to move (e.g., slide) through device body 214, however, engagement portions 202 (e.g., hooks) cannot extend as far from device body 214, due to the shorter length of backing portion 204 (FIG. 25B).

While various embodiments shown herein above are shown with all of engagement portions 202 (e.g., hooks) at relatively the same level or axial position, some embodiments of stabilizing members 200 (such as shown in FIG. 26A, FIG. 26B, and FIG. 26C) are configured with engagement portions 202 at different axial positions (e.g., axially staggered or offset from one another). For instance, hooked engagement portions 202 at different axial positions are advantageous for providing flexibility of the device anchoring location in different LAA anatomies.

Engagement Control

As described herein, some embodiments serve to address engagement control of a medical device with surrounding tissues to prevent movement of the device from its implant/target site. For instance, occlusion devices targeting the LAA require stabilizing members 200, desirably designed to reduce damage to the cardiac tissue. In some embodiments, at least one of penetration depth control, and/or extension control is additionally and advantageously imparted by the design of stabilizing member 200. It is therefore contemplated that the various features described in the following engagement control embodiments may be used in combination with one or more other engagement control features and/or in combination with one or more penetration depth control, and/or extension control features described elsewhere herein. Consequently, figures and embodiments showing a single engagement portion and/or a single backing portion with various features are also representative of embodiments having two or more engagement portions (e.g., multi-hooked stabilizing members) and/or embodiments having two or more backing portions (e.g., multi-legged stabilizing members) with the exemplified feature(s). In a similar manner, multi-engagement portion and/or multi-backing portion embodiments exemplifying various features are also representative of single engagement portion and/or single backing portion embodiments with the exemplified feature(s).

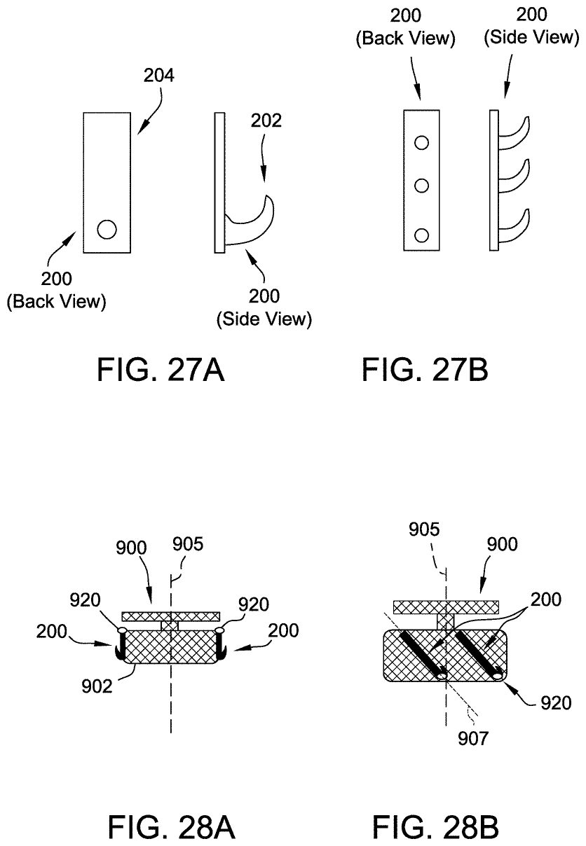

In an exemplary embodiment, stabilizing members 200 made from various polymers are injection molded and attached to the medical device. FIG. 27A illustrates an exemplary embodiment of an injection-molded stabilizing member with a single engagement portion 202 (e.g., a single hook), while FIG. 27B illustrates an exemplary embodiment of an injection-molded stabilizing member 200 with multiple engagement portions 202 (e.g., multiple hooks), in accordance with the present disclosure. In FIGS. 27A and 27B, engagement portions 202 are molded in various geometries as needed for appropriate tissue engagement, as described herein. The polymer material may include one or more of a variety of common materials suitable for injection molding including but not limited to, thermoplastics, bio-absorbable polymers, or shape memory polymers. In some embodiments, engagement portion 202 includes the means of attachment to the respective backing portion 204. Additionally or alternatively, engagement portion 202 is injection molded onto backing portion 204 (e.g., substrate). In some embodiments, engagement portion 202 is injection molded onto backing portion 204 (e.g., a strip of material), and stabilizing member 200 is coupled onto the medical device body by sewing. In some embodiments, backing portion 204 is comprised of any of a variety of materials depending upon requirements of the device and treatment of the target site. Materials for backing portion 204 include metal, stretchable fabric to allow engagement portions 202 to expand or contract with the medical device, or polymer sheets of the same or similar materials described for engagement portions 202 (e.g., hook(s)). In one exemplary embodiment, material for backing portion 204 is able to accommodate size change of the medical device from a collapsed first position to an expanded second position.

FIG. 28A and FIG. 28B show exemplary embodiments of attachment of stabilizing members 200 to a device body 902 (such as device body 214) of a medical device 900 in accordance with the present disclosure. As needed and depending on the embodiment, one or more stabilizing members 200 are attached to device body 902, each at a respective attachment point 920, such that each stabilizing member 200 has a single attachment point 920 (i.e., one attachment point 920 per stabilizing member 200). In some embodiments, stabilizing members 200 are attached to device body 902 in an orientation parallel to a longitudinal axis 905 of medical device 900, as shown in FIG. 28A. In other embodiments, stabilizing members 200 are attached to device body 902 at attachment point 920 such that each stabilizing member 200 is positioned at an angle (e.g., along a diagonal 907) relative to longitudinal axis 905, as shown in FIG. 28B.

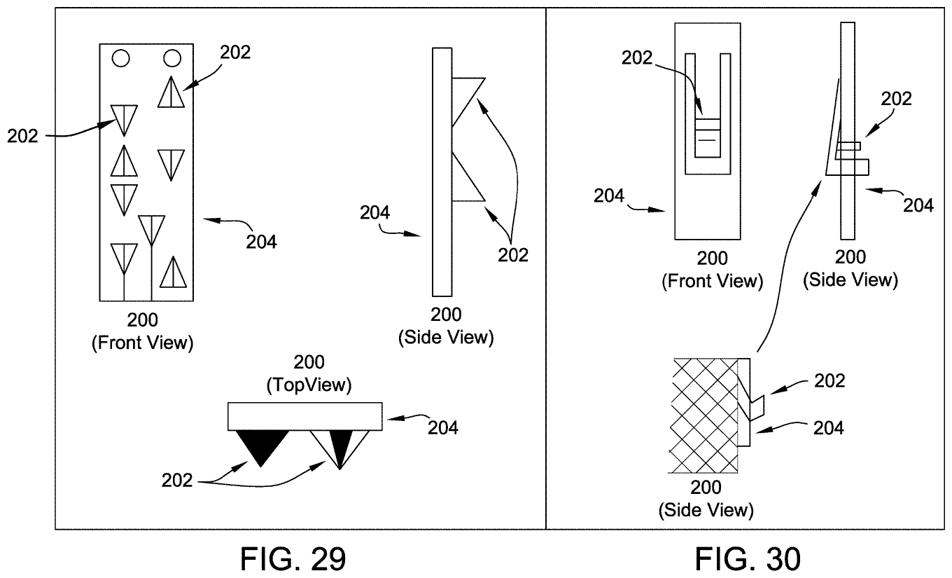

Turning now to FIG. 29, barbed engagement portions 202 are illustrated. In some embodiments, each barbed engagement portion 202 (also referred to as a “barb”) is a sharp projection extending from backing portion 204. For example, in some embodiments barbed engagement portion 202 is formed (e.g., formed from a jointed material, formed from injection molding, and/or cut, formed, and heat set) such that an outer tip of each joint is a sharp projection or barb. In some embodiments, when stabilizing member 200 includes a plurality of barbed engagement portions 202, barbed engagement portions 202 may be provided in more than one size and/or in more than one orientation. For example, as shown in the side view of stabilizing member 200 in FIG. 29, barbed engagement portions 202 have points that are generally pointing in different directions. The size of barbed engagement portions 202 may be defined by a height of the respective barb, such as a height from a base of the barb to the point of the barb. The size of barbed engagement portions 202 may be defined by an area of the base of the barb. As shown in the front view of stabilizing member 200 in FIG. 29, in some embodiments, barbed engagement portions 202 are staggered vertically and/or horizontally over an outward face of backing portion 204. In some embodiments, barbed engagement portions 202 are made of thermoplastic, bio-absorbable, shape memory, and/or injection-molded material such as polymers.

FIG. 30 is an exemplary embodiment of a stabilizing member 200 with a displaceable engagement portion 202 in accordance with the present disclosure. In the exemplary embodiment, engagement portion 202 (such as a hook or barb) is displaceable. For example, as shown in FIG. 30, upper and lower side views show engagement portion 202 generally aligned with backing portion 204 and engagement portion 202 generally protruding from backing portion 204, respectively. In the side view of stabilizing member 200 where engagement portion 202 is generally protruding from backing portion 204, engagement portion 202 has been displaced such that it extends radially outward from the body of the medical device. That is, FIG. 30 side views show a flexibility of displaceable engagement portion 202 (e.g., a tab) to retract for delivery, as well as to engage tissue on deployment and to go flush (e.g., lie even) with backing portion 204 and/or the body of the medical device. In some embodiments, a deployed configuration of the medical device causes displacement of the engagement portion 202. More particularly, in an exemplary embodiment, stabilizing members 200 are coupled to an exterior surface of the medical device body. The medical device body is formed from at least one braided layer, which engages an inward face of engagement portion and displaces engagement portion 202 (e.g., causes engagement portion 202 to extend radially outward past backing portion 204) when the medical device is in the deployed/expanded configuration. Embodiments described herein may include retractable and/or displaceable engagement portions 202. In some embodiments, stabilizing members 200 may be retractable and/or displaceable with the expanded or contracted configuration of the medical device.

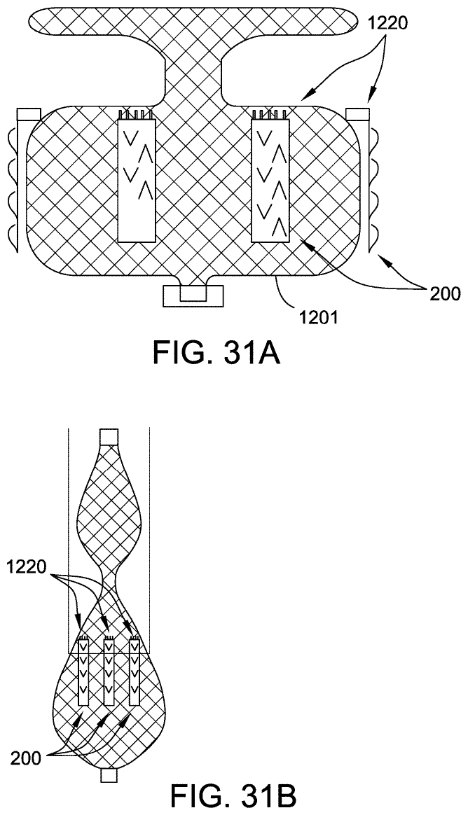

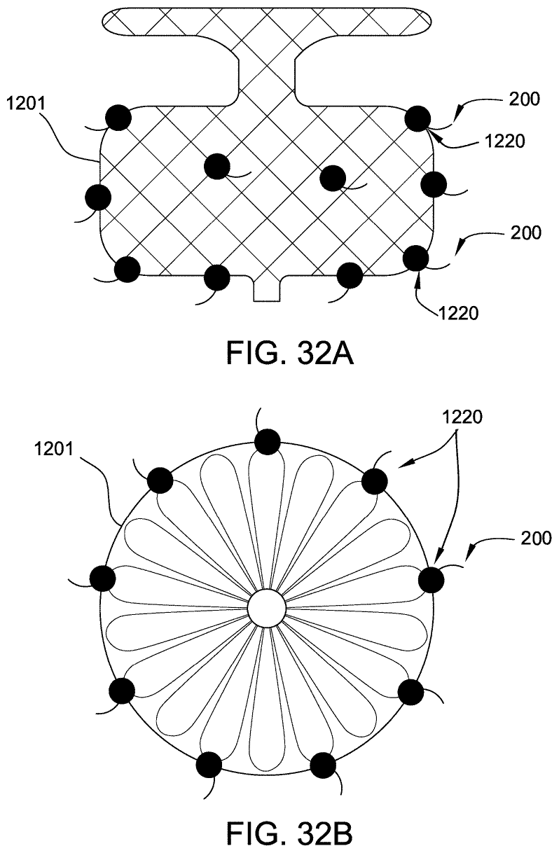

In FIGS. 31A-B and 32A-B, methods of attachment of stabilizing members 200 to a device body 1201 (such as device body 214 or 902) are disclosed. FIG. 31A is an exemplary embodiment of sewn-on placement of a stabilizing member 200 onto device body 1201 in accordance with the present disclosure. In some embodiments, backing portion 204 (when present) is sewn onto device body 1201 at attachment point 1220 (such as attachment point 920 shown in FIGS. 28A and 28B). As described further below, stabilizing members 200 attach to device body 1201 at one end (e.g., a distal or proximal end) of device body 1201, or in some embodiments at one end of a lobe portion and/or at one end of a disc portion of the device body 1201. Providing a single attachment point for each stabilizing member 200 allows the at least one braided layer of the device body to compress and move under the backing portion 204 of the engagement portion 202 (e.g., hooks/barbs). That is, a single suture location per each backing portion 204 allows the braid to elongate when the device is in a collapsed position for delivery, such as shown in FIG. 31B. As an example, in some embodiments a hooked engagement portion 202 may not have the same stretch-ability as the braided layer between expanded and contracted configurations of the medical device, therefore providing a single attachment point 1220 per stabilizing member 200 reduces the likelihood of adverse loading and delivery issues. Depending on the embodiment, single attachment point 1220 is located at a desired location on device body 1201, such as located at a top, middle, or bottom position of device body 1201. In other embodiments, for example when backing portion 204 is not present in stabilizing member 200, engagement portion 202 is sewn directly onto device body 1201. FIG. 32A and FIG. 32B show side view and top view embodiments, respectively, of injection-molded placement of stabilizing members 200 onto device body 1201. In some embodiments, stabilizing member 200 is injection molded (e.g., at backing portion 204, when present) onto device body 1201 at attachment point 1220. In other embodiments, for example when backing portion 204 is not present in stabilizing member 200, engagement portion 202 is injection-molded directly onto device body 1201.

In some embodiments, one or more stabilizing members 200 are sewn onto device body 1201 at a respective single attachment point 1220. In an exemplary embodiment, either a top or bottom of stabilizing member 200 is coupled (e.g., sewn) to device body 1201 at attachment point 1220, such that stabilizing member 200 can accommodate a size change of the medical device between a collapsed first configuration and an expanded second configuration. That is, a single attachment point 1220 (i.e., at either top or bottom of stabilizing member 200, not both) couples stabilizing member 200 to device body 1201 and does not inhibit expanding or collapsing of the medical device.

In other embodiments, one or more stabilizing members 200 are injection molded directly onto device body 1201, each at a respective single attachment point 1220 (FIG. 32A and FIG. 32B) using a combination of sewing and injection molding at each respective single attachment point 1220. That is, one attachment point 1220 per one stabilizing member 200.

While embodiments of the present invention have been described, it should be understood that various changes, adaptations and modifications may be made therein without departing from the spirit of the invention and the scope of the appended claims. For example, it is anticipated that the device body may comprise at least one disc and/or at least one lobe, where the lobe may be cylindrical, barrel shaped, concave, convex, tapered, or a combination of shapes without departing from the invention herein. Further, all directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. It is intended that all matter contained in the above description or shown in the accompanying drawings shall be interpreted as illustrative only and not limiting. Changes in detail or structure may be made without departing from the spirit of the disclosure as defined in the appended claims

Many modifications and other embodiments of the invention set forth herein will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A medical device for treating a target site, the medical device comprising:
a device body including at least one disc formed from a shape memory material; and
a plurality of stabilizing members coupled to the device body, each stabilizing member respectively comprising:
a backing portion coupled to the device body;
at least one engagement portion extending from an outward face of the backing portion, wherein the engagement portion extends radially outward from the device body; and
one or more features configured to control at least one of tissue penetration depth of the at least one engagement portion, extension of the stabilizing member from the device body, and tissue engagement of the at least one engagement portion.

2. The medical device of claim 1, wherein the device body further includes a lobe formed from a shape memory material.

3. The medical device of claim 1, wherein each stabilizing member is formed from the group consisting of at least one wire, a laser cut nitinol form, an injection molded shape memory polymer, an injection molded bio-absorbable polymer, and combinations thereof.

4. The medical device of claim 1, wherein each stabilizing member is coupled to the device body such that an entirety of the at least one engagement portion and at least a portion of the backing portion is located external to the device body.

5. The medical device of claim 1, wherein each stabilizing member is coupled to the device body such that an entirety of the at least one engagement portion is located external to the device body and at least a portion of the backing portion is located internal to the device body.

6. The medical device of claim 1, wherein the one or more features comprises at least one penetration stop defining a tissue penetration depth limit at a location where the at least one penetration stop adjoins the backing portion.

7. The medical device of claim 1, wherein each engagement portion is selected from at least one hook and at least one barb.

8. The medical device of claim 1, wherein the shape memory material comprises a braided mesh fabric, and wherein each stabilizing member is coupled to an interior surface of the device body.

9. The medical device of claim 1, wherein the shape memory material comprises a braided mesh fabric, and wherein each stabilizing member is coupled to an exterior surface of the device body.

10. The medical device of claim 1, wherein each stabilizing member is coupled to the device body by at least one of being sewn directly onto the device body and being injection molded directly onto the device body.

11. The medical device of claim 1, wherein each stabilizing member further comprises a rotation restraint selected from the group consisting of a tail, a loop and a T-bar.

12. The medical device of claim 1, wherein the engagement portion curves from the backing portion to form a recess angle, such that the engagement portion does not extend linearly straight from the backing portion.

13. The medical device of claim 12, further comprising:
an eyelet coupled to the backing portion, wherein the eyelet is directly attached to the device body.

14. The medical device of claim 1, wherein the one or more features comprises an attachable feature coupled to the backing portion, and wherein the attachable feature is directly attached to the device body.

15. The medical device of claim 14, wherein the attachable feature is selected from the group consisting of an eyelet, a protuberance, a node, a knob, a projection, and a ledge.

16. The medical device of claim 1, wherein the one or more features comprises an arresting feature coupled to the backing portion, and wherein the arresting feature is not directly attached to the device body.

17. The medical device of claim 16, wherein the arresting feature is selected from the group consisting of a protuberance, a node, a knob, a projection, and a ledge.

18. The medical device of claim 1, wherein each stabilizing member comprises two backing portions connected by a section of material.

19. The medical device of claim 18, wherein the two backing portions are crossed.

20. The medical device of claim 1, wherein the backing portion is a stent-like structure.

21. The medical device of claim 1, wherein the at least one engagement portion comprises at least two engagement portions, and wherein the at least two engagement portions are located at different axial positions relative to the device body.

22. The medical device of claim 1, wherein each stabilizing member comprises a backing portion and at least two engagement portions, and wherein a maximum extension length is defined by a bifurcation formed at a location where the at least two engagement portions adjoin the backing portion.

23. A delivery system comprising:
a medical device comprising:
a device body including at least one disc formed from a shape memory material; and
a plurality of stabilizing members coupled to the device body, each stabilizing member comprising:
a backing portion coupled to the device body;
at least one engagement portion extending from an outward face of the backing portion, wherein the at least one engagement portion extends radially outwardly from the device body; and
one or more features configured to control at least one of tissue penetration depth of the at least one engagement portion, extension of the stabilizing member from the device body, and tissue engagement of the at least one engagement portion; and
a delivery sheath configured to retain and recapture the medical device during deployment of the medical device to a target site.

24. The delivery system of claim 23, wherein the device body further includes a lobe formed from a shape memory material.

25. The delivery system of claim 23, wherein each stabilizing member is formed from the group consisting of at least one wire, a laser cut nitinol form, an injection molded shape memory polymer, an injection molded bio-absorbable polymer, and combinations thereof.

26. The delivery system of claim 23, wherein each stabilizing member is coupled to the device body such that an entirety of the at least one engagement portion and at least a portion of the backing portion is located external to the device body.

27. The delivery system of claim 23, wherein each stabilizing member is coupled to the device body such that an entirety of the at least one engagement portion is located external to the device body and at least a portion of the backing portion is located internal to the device body.

28. The delivery system of claim 23, wherein the one or more features comprises at least one penetration stop defining a tissue penetration depth limit at a location where the at least one penetration stop adjoins the backing portion.

29. The delivery system of claim 23, wherein each engagement portion is selected from at least one hook and at least one barb.

30. The delivery system of claim 23, wherein the shape memory material comprises a braided mesh fabric, and wherein each stabilizing member is coupled to an interior surface of the device body.

31. The delivery system of claim 23, wherein the shape memory material comprises a braided mesh fabric, and wherein each stabilizing member is coupled to an exterior surface of the device body.

32. The delivery system of claim 23, wherein each stabilizing member is coupled to the device body by at least one of being sewn directly onto the device body and being injection molded directly onto the device body.

33. The delivery system of claim 23, wherein each stabilizing member further comprises a rotation restraint selected from the group consisting of a tail, a loop and a T-bar.

34. The delivery system of claim 23, wherein the engagement portion curves from the backing portion to form a recess angle, such that the engagement portion does not extend linearly straight from the backing portion.

35. The delivery system of claim 34, further comprising:
an eyelet coupled to the backing portion, wherein the eyelet is directly attached to the device body.

36. The delivery system of claim 23, wherein the one or more features comprises an attachable feature coupled to the backing portion, and wherein the attachable feature is directly attached to the device body.

37. The delivery system of claim 36, wherein the attachable feature is selected from the group consisting of an eyelet, a protuberance, a node, a knob, a projection, and a ledge.

38. The delivery system of claim 23, wherein the one or more features comprises an arresting feature coupled to the backing portion, and wherein the arresting feature is not directly attached to the device body.

39. The delivery system of claim 38, wherein the arresting feature is selected from the group consisting of a protuberance, a node, a knob, a projection, and a ledge.

40. The delivery system of claim 23, wherein each stabilizing member comprises two backing portions connected by a section of material.

41. The delivery system of claim 40, wherein the two backing portions are crossed.

42. The delivery system of claim 23, wherein the backing portion is a stent-like structure.

43. The delivery system of claim 23, wherein the at least one engagement portion comprises at least two engagement portions, and wherein the at least two engagement portions are located at different axial positions relative to the device body.

44. The delivery system of claim 23, wherein each stabilizing member comprises a backing portion and at least two engagement portions, and wherein a maximum extension length is defined by a bifurcation formed at a location where the at least two engagement portions adjoin the backing portion.
